(19) United States
(12) Patent Application Publication
(10) Pub. No.: US 2025/0170168 A1
(43) Pub. Date: May 29, 2025

(54) High Concentration H2 Aqueous Solution And Its Use

(71) Applicant: BLUE HYDROGEN SCIENCE S.R.L, Belluno (IT)

(72) Inventors: Raffaele INGENITO, Roma (IT); Giorgio MATTETTI, Belluno (IT); Giuliano TALO', Belluno (IT)

(21) Appl. No.: 18/851,368

(22) PCT Filed: Mar. 29, 2023
(86) PCT No.: PCT/EP2023/058095

§ 371 Date: Sep. 26, 2024

(30) Foreign Application Priority Data
Mar. 31, 2022 (WO) PCT/IT2022/000016

(51) Int. Cl. A61K 33/00; A61K 9/08; A61K 33/06; A61M 35/00; A61P 39/06

(52) U.S. Cl. CPC A61K 33/00; A61K 9/08; A61K 33/06; A61M 35/30; A61P 39/06

(57) ABSTRACT

The present invention concerns an aqueous solution comprising molecular hydrogen and at least one salt, wherein said solution has an ionic strength in the range from 0.001 mol/l to 1 mol/l and comprises ascorbic acid or a salt thereof. The solution can be used for ameliorating the activation of cell mechanisms that reduce cellular oxidative stress and cell toxicity by transdermal administration to a subject and for use in treating oxidative stress-related diseases.

### FIELD OF THE INVENTION

The present invention concerns an aqueous solution comprising molecular hydrogen and the cosmetic use of the aqueous solution for ameliorating the activation of cell mechanisms that reduce cellular oxidative stress and cell toxicity by transdermal administration to a subject. The aqueous solution of the invention can be used in treating oxidative stress-related diseases by reducing cellular oxidative stress and reducing cell toxicity by transdermal administration.

### STATE OF THE ART

It is known that oxidative stress, i.e. a quantity of reactive oxygen species (ROS) exceeding physiological levels, causes physical damage that becomes manifest in various clinical conditions. There is a large number of diseases of the human body in which free chemical radicals, but particularly reactive oxygen species, are generally involved. Radicals are taken in from the environment (air, food, water) or formed by the body itself. It is the biological function of ROS in the body to kill microbes (bacteria, fungi, viruses). But these radicals are themselves harmful to the body and can modify endogenous substances, such as lipids, hyaluron, and proteins. In this process, some substances are formed, e.g. oxidized phospholipids, which cause other inflammatory reactions by activating, for example, toll-like receptors (TLR2 and TLR4) and thus ultimately cause an additional release of ROS.

It is known from prior art that hydrogen, if inhaled or dissolved in water, is capable of rendering free radicals harmless in the body.

The size of the hydrogen molecule is the smallest of all substances, and it easily penetrates the cell membrane. There is no concern about side effects when hydrogen that is stable and inactive is put into the body. Hydrogen-rich water removes reactive oxygen species (ROS).

In Ryoko Asada et al “Effects of hydrogen-rich water bath on visceral fat and skin blotch, with boiling-resistant hydrogen bubbles”, MEDICAL GAS RESEARCH, Vol. 9, no. 2, 30nJune 2009, the use of hydrogen-dissolved water in the treatment of oxidation stress-related skin troubles is disclosed. In this article the hydrogen-bath utilization improved cosmetic effects such as skin-blotch repression and the visceral-fat-based slimming effects. Specifically, the subjects were treated by immersion of their body in warm (41° C.) hydrogen-rich water bath for 10 minutes once a day for 1 month and it was shown that a hydrogen-rich water bath that produces abundant dissolved hydrogen helps hydrogen permeate into the body from the skin and improves the visceral fat area, the lipid metabolic markers and the skin blotch being attributed to boiling-resistant hydrogen bubbles through ROS removal. The potential anti-fatigue and performance benefits of hydrogen-rich water have led to increased research interest over the past 5 years among the athletes. The physiological and perceptual responses to a protocol of incremental aerobic and anaerobic exercise, with subsequent administration of transcutaneous hydrogen post exercise and the monitoring of any side effects and adverse events (AEs) were evaluated. In it also known that transcutaneous hydrogen-rich water improved ventilatory responses, perceptive and lactate to physical exercise, aerobic and anaerobic, in absolute conditions safety conditions.

It is felt the need of methods and apparatuses for delivering molecular hydrogen in a efficacious way.

It is therefore object of the present invention the development of an effective use of hydrogen water for physiologically beneficial purposes for the organism of a human or an animal subject, particularly detoxifying purposes, antioxidant purposes to relieve oxidative stress of the organism, sporting and therapeutic purposes on both human and animal subjects.

### SUMMARY OF THE INVENTION

The above object has been achieved by an aqueous solution comprising molecular hydrogen and at least one salt, wherein said solution has an ionic strength in the range from 0.001 mol/I to 1 mol/I and comprises ascorbic acid or a salt thereof.

The inventors noted that the solution of the invention was capable to activate cell mechanisms that reduce cellular oxidative stress measured in the amount of super oxide dismutase (SOD) and allow the reduction of plasmatic acidosis measured in the amount of lactase in a very efficacious way.

In a further aspect therefore the invention relates to a cosmetic use of the aqueous solution of the invention for ameliorating the activation of cell mechanisms that reduce cellular oxidative stress and cell toxicity by transdermal administration.

Therefore according to the invention, the cosmetic use showed surprising effects on subjects practicing a sport activity, preferably an athlete.

In a further aspect the invention relates to the aqueous solution of the invention for use in treating oxidative stress-related diseases by reducing cellular oxidative stress and reducing cell toxicity by transdermal administration

Without being bound to any theory and as demonstrated by the experimental part, the inventors deem that the specific compounds of the aqueous solution of the invention allow to provide a very high concentration of molecular hydrogen that passes through the skin and is capable to intervene on cellular mechanisms responsible of the oxidative stress and cell toxicity

In another aspect the invention relates to a method for administering hydrogen by transdermal or percutaneous administration, including the step of providing an aqueous solution of the invention by introducing hydrogen in water contained in one or in a plurality of dipping vessels, suitable for dipping the whole or parts of the body thereinto of a human subject, preferably hands and/or feet, or of an animal subject.

In said aqueous solution of the invention, hydrogen is kept in an effective concentration for the permeation through the skin of a human subject, including hands and/or feet and other body parts, or of an animal subject, through the dipping of the whole or parts of the body of the human or animal subject itself, whereby the aqueous solution of hydrogen and salts, when kept preferably at a temperature falling in the range between 10° C. and 55° C., is a hydrogen water effective for administering transdermal hydrogen having a physiologically beneficial activity for the organism of the human or the animal subject, including detoxifying activity, antioxidant activity to relieve oxidative stress of the organism, for therapeutic use and for sporting activity.

### DESCRIPTION OF THE FIGURES

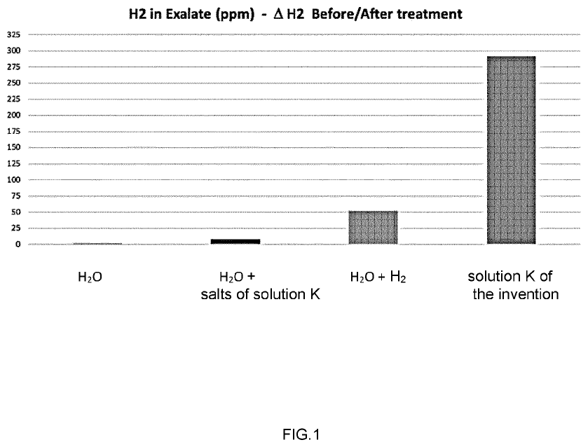

In FIG. 1 the results of Example 2 are reported.

### DETAILED DESCRIPTION OF THE INVENTION

The present invention concerns an aqueous solution comprising molecular hydrogen and at least one salt, wherein said solution has an ionic strength in the range from 0.001 mol/I to 1 mol/I and comprises ascorbic acid or a salt thereof.

When in the present invention the term “molecular hydrogen” is used, it is intended hydrogen gas dissolved in the water and present in the form of bubbles in micro and nano amounts.

The molecular hydrogen is present in the solution in an amount (concentration) in the range from 0.05 ppm (mg/L) to 1.2 ppm (mg/L), more preferably from 0.4 ppm (mg/L) to 0.9 ppm (mg/L).

The aqueous solution of the invention comprises at least one salt.

The at least one salt is preferably selected from the group consisting of NaCl (sodium chloride); Na(OH) (sodium hydroxide); NaHCO₃ (sodium bicarbonate); Na₂[CO₃](sodium carbonate); NaC₆HzO₆ (sodium ascorbate); Na₃C₆H₅O₇(sodium citrate); NaH (sodium hydride); NaHSO₄ (sodium hydrogen sulfate); Na₂SO₄ (sodium sulfate); Na₃PO₃ (sodium phosphate); NaHPO₃ (sodium dihydrogen phosphate); Na₂PO₃ (sodium hydrogen phosphate); NaO₂CCH(OH)CH(OH)CO₂H (monobasic sodium tartrate); NaO₂CCH(OH)CH(OH)CO₂Na (sodium tartrate); NaOCOCH(OH)CH(OH)COOK (potassium sodium tartrate); CH₂(COONa)₂ (disodium malonate); KCl (potassium chloride); K(OH) (potassium hydroxide); KHCO₃ (potassium bicarbonate); K₂[CO₃](potassium carbonate); KC₆H₇O₆ (potassium ascorbate); K₃C₆H₅O₇ (potassium citrate); KH (potassium hydride); KHSO₄ (potassium hydrogen sulfate); K₂SO₄ (potassium sulfate); K₃PO₃ (potassium phosphate); KHPO₃ (potassium dihydrogen phosphate); K₂PO₃ (potassium hydrogen phosphate); KOCOCH(OH)CH(OH)COOH (monobasic potassium tartrate); KOCOCH(OH)CH(OH)COK (potassium tartrate); KOCOCH(OH)CH(OH)COONa (potassium sodium tartrate); MgCl₂ (magnesium chloride); MgO (magnesium oxide); Mg(OH)₂ (magnesium hydroxide); MgH₂ (magnesium hydride); Mg[HCO₃]₂(magnesium bicarbonate); MgCO₃ (magnesium carbonate); Mg₃[C₆H₅O₇]₂(magnesium citrate); MgC₁₀H₁₂N₂O₆ (magnesium pidolate); MgSO₄ (magnesium sulfate); Mg(HSO₄)₂ (magnesium hydrogen sulfate); CaCl₂ (calcium chloride); CaO (calcium oxide); Ca(OH)₂ (calcium hydroxide); CaH₂ (calcium hydride); Ca[HCO₃]₂ (calcium bicarbonate); CaCO₃ (calcium carbonate); Ca₃[C₆H₅O₇]₂ (calcium citrate); CaSO₄ (calcium sulfate); Ca(HSO₄)₂ (calcium hydrogen sulfate); Ca₃(PO₄)₂ (calcium phosphate); CaHPO₄ (calcium hydrogen phosphate); Ca(H₂PO₄)₂ (calcium dihydrogen phosphate); MnCl₂ (manganese chloride); MnO (manganese oxide); Mn(OH)₂ (manganese hydroxide); Mn(SO₄) (manganese sulfate); Mn[HCO₃]₂ (manganese hydrogen carbonate); MnCO₃ (manganese carbonate); Mn₃[C₆H₅O₇]₂ (manganese citrate); Mn₃ (PO₄)₂ (manganese phosphate); and mixtures thereof.

More preferably the at least one salt is a monohydrogen carbonate, a bicarbonate or a chloride of an alkaline or alkaline earth metal, still more preferably it is dihydrogen carbonate or a chloride of an alkaline metal.

In a preferred embodiment the at least one salt is selected from the group consisting of sodium bicarbonate, potassium carbonate, magnesium chloride and a mixture thereof.

The at least one salt of the invention is preferably present in the aqueous solution in an amount in the range from 10 mM to 800 mM, more preferably 50 mM to 400 mM.

The aqueous solution of the invention comprises ascorbic acid or a salt thereof. The ascorbic acid or its salt is preferably in an amount in the range from 10 mM to 300 mM.

Without being bound to any theory the inventors deem that the ascorbic acid or its salt is essential to guarantee that the molecular hydrogen is efficaciously administered to the cells when a subject is in contact with the aqueous solution of the invention.

The aqueous solution of the invention has an ionic strength in the range from 0.001 mol/I to 1 mol/I, preferably from 0.0075 mol/L to 0.5 mol/L, more preferably 0.250 mol/L.

The aqueous solution of the invention can also comprise other ingredients/substances, such as vitamins and/or essential oils.

The aqueous solution of the invention has preferably a pH value in the range from 6 to 11.

The inventors noted that the solution of the invention in view of the essential ingredients and of the specific ionic strength was capable to activate cell mechanisms that reduce cellular oxidative stress measured in the amount of super oxide dismutase (SOD) and allow the reduction of plasmatic acidosis measured in the amount of lactase in a very efficacious way.

In a further aspect therefore the invention relates to a cosmetic use of the aqueous solution of the invention for ameliorating the activation of cell mechanisms that reduce cellular oxidative stress and cell toxicity by transdermal administration.

Therefore according to the invention, the cosmetic use showed surprising effects on subjects practicing a sport activity, preferably an athlete.

In a further aspect the invention relates to the aqueous solution of the invention for use in treating oxidative stress-related diseases by reducing cellular oxidative stress and reducing cell toxicity by transdermal administration Without being bound to any theory and as demonstrated by the experimental part, the inventors deem that the specific compounds of the aqueous solution of the invention with the specific strength allow to provide a very high concentration of molecular hydrogen that passes through the skin and is capable to intervene on cellular mechanisms responsible of the oxidative stress and cell toxicity

The aqueous solution of the invention is preferably at a temperature in the range from 37° C. to 50° C. when used in the cosmetic use or in a medical treatment of the invention.

In another aspect the invention relates to a method for administering hydrogen by transdermal or percutaneous administration, including the step of providing an aqueous solution of the invention by introducing hydrogen in water contained in one or in a plurality of dipping vessels, suitable for dipping the whole or parts of the body thereinto of a human subject, preferably hands and/or feet, or of an animal subject.

In said aqueous solution of the invention, hydrogen is kept in an effective concentration for the permeation through the skin of a human subject, including hands and/or feet and other body parts, or of an animal subject, through the dipping of the whole or parts of the body of the human or animal subject itself, whereby the aqueous solution of hydrogen and salts, when kept preferably at a temperature falling in the range between 10° C. and 55° C., is a hydrogen water effective for administering transdermal hydrogen having a physiologically beneficial activity for the organism of the human or the animal subject, including detoxifying activity, antioxidant activity to relieve oxidative stress of the organism, for therapeutic use and for sporting activity.

The invention also relates to an apparatus for carrying out the treatment disclosed above or for performing the use above reported, comprising a hydrogen generator, also known as ‘hydrogenator’; one or a plurality of use stations, arranged in proximity and/or remotely with respect to the hydrogen generator and each one including one or a plurality of dipping vessels, suitable for dipping the whole or parts of the body of a human subject, including hands and/or feet, or of an animal subject, suitable for being loaded with water; a piping for conveying input load water into the one or each one of the plurality of dipping vessels; a piping for conveying output unloading water from the one or each one of the plurality of dipping vessels; a piping for conveying output hydrogen from the hydrogen generator, arranged for introducing the hydrogen into the water loaded into the one or each one of the plurality of dipping vessels, generating a hydrogen aqueous solution therein; one or a plurality of dispensers of at least one salt; a conveyor arranged for collecting the salts dispensed therefrom and conveying them to the one or each one of the plurality of dipping vessels.

The apparatus can further preferably comprise a control system for controlling the dispensers of at least one salt, suitable for so controlling the at least one salt that they dispense in said conveyor means in specific amounts. Such specific amounts should preferably guarantee dissolution of the salts in the dipping vessels loaded with water so as the aqueous solution together with molecular hydrogen and the ascorbic acid or its salt has:
a pH value in the range from 6 to 11; and
an ionic strength value in the range from 0.001 moles/litre to 1 mole/litre.

The apparatus further can include a control system for controlling the temperature of the hydrogen aqueous solution in the dipping vessels to a temperature in the range from 10° C. to 55° C.

In a preferred embodiment of the invention the control system for controlling the temperature of the aqueous solution of the invention in the dipping vessels controls to a temperature in the range from 37° C. to 50° C.

It is further envisaged that the apparatus further preferably comprises one or a plurality of dispensers of one or a plurality of vitamins and/or essential oils; a conveyor arranged for collecting the vitamins and/or essential oils dispensed therefrom and conveying them to the one or each one of the plurality of dipping vessels.

The apparatus further can comprise a control system for controlling the dispensers of one or a plurality of vitamins and/or essential oils.

The apparatus further can be equipped with sensors and recorders of body dipping times, pH, ionic strength and the temperature, and variations thereof, in the aqueous solution in the dipping vessels and/or in communication vessels therebetween, and with a programmable data processor, in functional communication therebetween. The programmable data processor is in functional communication with the control system and is programmed for carrying out the method of this invention disclosed above.

In a further aspect the invention concerns also a computer program including code means that, when run on the aforesaid programmable data processor, are suitable for carrying out the method/use of this invention disclosed above, and a physical data medium configured to store the computer program.

In a still further aspect the invention concerns also an armchair to be associated to the apparatus including a seat and two armrests, equipped with bowls arranged on one or both armrests, and with bowls arranged below the seat. So, the bowls are suitable as dipping vessels, respectively suitable for dipping hands and/or feet thereinto by a human subject placed on the seat. So, the armchair is suitable for being a patient/subject station for treating the hands and/or the feet of a patient/subject by means of the method of this invention disclosed above. The armchair further can be equipped with a console for controlling the apparatus of this invention disclosed above.

Various embodiments and aspects of the present invention as delineated hereinabove and as claimed in the claims section below find experimental support in the following examples.

### EXAMPLES

### Example 1. Preparation of the Aqueous Solution of the Invention

Different aqueous solutions were prepared by using the following ingredients.
Aqueous solution A: 15 g of sodium bicarbonate; 15 g of potassium carbonate, 15 g of magnesium chloride and 15 g of ascorbic acid
Aqueous solution B: 30 g of sodium bicarbonate; 30 g of potassium carbonate, 30 g of magnesium chloride and 15 g of ascorbic acid
Aqueous solution E: 45 g of sodium bicarbonate; 15 g of potassium carbonate, 45 g of magnesium chloride and 30 g of ascorbic acid
Aqueous solution F: 15 g of sodium bicarbonate; 30 g of potassium carbonate, 45 g of magnesium chloride and 15 g of ascorbic acid
Aqueous solution G: 15 g of sodium bicarbonate; 30 g of potassium carbonate, 45 g of magnesium chloride and 45 g of ascorbic acid
Aqueous solution H: 30 g of sodium bicarbonate; 15 g of potassium carbonate, 45 g of magnesium chloride and 30 g of ascorbic acid
Aqueous solution I: 15 g of sodium bicarbonate; 30 g of magnesium chloride and 15 g of ascorbic acid
Aqueous solution J: 45 g of potassium carbonate, 60 g of magnesium chloride and 30 g of ascorbic acid
Aqueous solution K: 45 g of sodium bicarbonate; 45 g of magnesium chloride and 15 g of ascorbic acid

In a vessel comprising water in amount of 51 the salts were added thus obtaining the solution above reported.

Hydrogen gas was then introduced in the vessel thus obtaining an amount of molecular hydrogen in the range from 0.05 ppm to 1.5 ppm.

The final pH of the solutions were in the range from 7 to 9 as measured with bench pHmeter VioLab, specifically:
Aqueous Solution A: pH was 7.7 before the introduction of H₂ and 7.9 after the introduction of H₂
Aqueous solution B: pH was 7.88 before the introduction of H₂ and 8.05 after the introduction of H₂
Aqueous solution E: pH was 7.8 before the introduction of H₂ and 7.9 after the introduction of H₂
Aqueous solution F: pH was 7.8 before the introduction of H₂ and 7.9 after the introduction of H₂
Aqueous solution G: pH was 7.54 before the introduction of H₂ and 7.9 after the introduction of H₂
Aqueous solution H: pH was 7.88 before the introduction of H₂ and 7.9 after the introduction of H₂
Aqueous solution I: pH was 7.71 before the introduction of H₂ and 7.71 after the introduction of H₂
Aqueous solution J: pH was 7.88 before the introduction of H₂ and 8.05 after the introduction of H₂
Aqueous solution K: pH was 7.3 before the introduction of H₂ and 8.2 after the introduction of H₂

The ionic strength (I) of the single solutions was measured immediately at the addition of the salts and after the introduction of H₂ with direct measurement of Total Dissolved Solids Solutions (TDS) by using the S700 conductivity meter (Mettler Toledo) and the results were expressed in mol/liter by conversion the relationship equation (I (mol/L)≈2.5 10−5×TDS (mg/L) and are reported in Table 1:

TABLE 1

| | Ionic Strength (mol/liter) | | |
|---|---|---|---|
| | | Water plus solution | Water plus solution of the |
| Solution | Water | without H₂ | invention plus H₂ |
| A | 0.0075 | 0.0975 | 0.0975 |
| B | 0.0075 | 0.1975 | 0.235 |
| E | 0.0075 | 0.24 | 0.25 |
| F | 0.0075 | 0.21 | 0.22 |
| G | 0.0075 | 0.2125 | 0.225 |
| H | 0.0075 | 0.22 | 0.249 |
| I | 0.0075 | 0.0925 | 0.1 |
| J | 0.0075 | 0.235 | 0.25 |
| K | 0.0075 | 0.210 | 0.2225 |

Solution A: 0.0975 mol/L
Solution B: 0.235 mol/L
Solution E: 0.25 mol/L
Solution F: 0.22 mol/L
Solution G: 0.225 mol/L
Solution H: 0.249 mol/L
Solution 1 0.1 mol/L
Solution J: 0.250 mol/L
Solution K: 0.2225 mol/L

### Example 2

The solutions prepared in Example 1 were used and tested for evaluating the molecular hydrogen passed through percutaneous administration of subjects.

Firstly, the amounts of the dissolved molecular hydrogen in the vessels were evaluated after 24 hours and then 2 subjects (one man and one woman) were requested to dip feet and hands into the different vessels containing the different solutions.

The subjects were hence evaluated with a breath test in order to quantify how much molecular hydrogen was passed to the body through the aqueous solutions of the invention.

The results are reported in the following Table 2.

TABLE 2

| | | | | Breath test | |
|---|---|---|---|---|---|
| | | | Breath | post- | |
| | | | TEST | treatment | |
| | | pH | pre- | with the | |
| | | after 20 | treatment | solution | Difference |
| Subject | Solution | min | (ppm) | (ppm) | in ppm H2 |
| 1 | A | 8.56 | 0 | 27 | 27 |
| | B | 8.22 | 6 | 392 | 386 |
| | E | 7.88 | 17 | 430 | 413 |
| | F | 8.39 | 22 | 352 | 330 |
| | G | 8.39 | 23 | 228 | 205 |
| | H | 8.39 | 17 | 431 | 414 |
| | I | 7.71 | 18 | 340 | 322 |
| | J | 8.39 | 20 | 213 | 193 |
| | K | 8.2 | 2 | 294 | 292 |
| 2 | A | 8.56 | 11 | 52 | 41 |
| | B | 8.22 | 49 | 398 | 349 |
| | E | 7.88 | 3 | 185 | 182 |
| | F | 8.39 | 14 | 134 | 120 |
| | G | 8.39 | 11 | 81 | 70 |
| | H | 8.39 | 8 | 158 | 150 |
| | I | 7.71 | 3 | 97 | 94 |
| | J | 8.39 | 5 | 203 | 198 |
| | K | 8.2 | 3 | 295 | 292 |

As it evident from above the solutions of the invention guarantee the passage of the molecular hydrogen to the body. Better results were obtained with solutions B, E, F, H and K, with a very high amount of molecular hydrogen.

As above explained the inventors deem that the presence of the salts and of ascorbic acid or its salt in a water solution having a ionic strength in the range from 0.001 mol/I to 1 mol/I guarantee the stability of the hydrogen aqueous solution and allows the transportation of the molecular hydrogen to the cells. It was evident that the solutions with high concentration of molecular hydrogen but in absence of the essential ingredients of the solution of the invention the molecular hydrogen transported to the cells was evidently lower.

In FIG. 1 the results of the amount of H₂ as measured with the breath test or subjects 1 and 2 are reported. Specifically, the evaluation was done with =only water, −water+salts of solution K (Table 2), −water+H₂ and −water+salts+ascorbic acid+H₂ (solution K of the invention). The aqueous solution K was: 45 g of sodium bicarbonate; 45 g of magnesium chloride and 15 g of ascorbic acid; pH was 7.3 before the introduction of H₂ and 8.2 after the introduction of H₂.

TABLE 3

| | H2 for 20 min | | | |
|---|---|---|---|---|
| | | H2O + | H2O | H2O + |
| | | Solution | + | Solution |
| H2 in Exalate | H2O | K | H2 | K + H2 |
| Before Treatment | 6 | 10 | 2 | 2 |
| After Treatment | 8 | 18 | 54 | 294 |
| Δ Before/After | +2 ppm | +8 ppm | +52 ppm | +292 ppm |
| treatment | | | | |

It is evident from the graph of FIG. 1 the solution of the invention allowed surprisingly the passage of H₂ through the cells.

This high amount of molecular hydrogen passed to the cells allowed to reduce the oxidative stress as direct linked to some biomarkers such as the Superoxide Dismutase concentration in plasma (SOD) as well as the BAP (Antioxidant Biological Potential) which can be directly measured by blood sampling before and after the H₂ treatment as also shown in the example 3. In this way it was possible to demonstrate that this approach is a very effective method in the treatment of stress oxidative reduction.

### Example 3. Reduction of Superoxide Dismutase and Antioxidant Biological Potential

Oxidative stress is caused by the overproduction of Reactive Oxygen Species (ROS) and under physiological conditions, ROS are involved in processes, including cellular homeostasis, modulation of cellular metabolism, signaling and redox state, and being used by the immune system to inactivate viruses and inhibit bacterial growth. However, excess ROS production under oxidative stress conditions contributes to many pathological conditions and diseases, including cancer, neurological disorders, atherosclerosis, hypertension, ischemia/perfusion, diabetes, acute respiratory distress syndrome, idiopathic pulmonary fibrosis, chronic obstructive pulmonary disease, and asthma. The human body is equipped with a variety of enzymatic antioxidants that serve to counterbalance the effect of oxidants. One the major player in the counteract the oxidative stress is the enzyme Supeoxide Dismutase (SOD) which is a scavenger of superoxide radicals. It has been demonstrated that the SOD is an effective biomarker of the physiological oxidative stress, meaning that its concentration is naturally higher in oxidation conditions, independently from the causes. Therefore, in case of correct balance of oxidative stress the concentration of SOD has to normalize to basic levels (Ref. J Toxicol Environ Health.1996 Jun. 7; 48(2):107-19.doi: 10.1080/009841096161366; Clin Med. 2022 Aug. 31; 11(17):5131. doi: 10.3390/jcm11175131; World Allergy Organ J. 2012 January; 5(1): 9-19; Hypertens Res. 2011 Sep.; 34(9):1041-5).

In order to demonstrate the efficiency of transcutaneous administration of H₂ in reducing the oxidative stress loading in non-professional athletes at maximum effort (the so called Sports Paradox), 10 non-professional athletes were treated for 5 days to transcutaneous H2 activity for 20 min/day through the solutions of the invention, specifically solution K and by plasma sampling SOD and BAP (Antioxidant Biological Potential) concentrations before and after the treatment according to the invention were measured.

The parameters (BAP and SOD) were measured by blood sampling at the time of max physical effort of athletes and the mean value of variation for the ten athletes is reported in the table 4 below:

TABLE 4

| Oxidative Stress Plasma Biomarker | % change |
|---|---|
| BAP | +7% ↑ |
| SOD | −13% ↓ |

As it can be seen both the biomarkers ameliorated through the treatment of the invention.

1. An aqueous solution comprising molecular hydrogen and at least one salt, wherein said solution has an ionic strength in the range from 0.001 mol/1 to 1 mol/1 and comprises ascorbic acid or a salt thereof.

2. The aqueous solution of claim 1, wherein the molecular hydrogen is present in the solution in an amount (concentration) in the range from 0.05 ppm (mg/L) to 1.2 ppm (mg/L).

3. The aqueous solution of claim 1, wherein the at least one salt is selected from the group consisting of NaCl (sodium chloride); Na(OH) (sodium hydroxide); NaHCO₃ (sodium bicarbonate); Na₂[CO₃](sodium carbonate); NaC₆H₇O₆ (sodium ascorbate); Na₃C₆H₅O₇ (sodium citrate); NaH (sodium hydride); NaHSO₄ (sodium hydrogen sulfate); Na₂SO₄ (sodium sulfate); Na₃PO₃ (sodium phosphate); NaHPO₃ (sodium dihydrogen phosphate); Na₂PO₃ (sodium hydrogen phosphate); NaO₂CCH(OH)CH(OH)CO₂H (monobasic sodium tartrate); NaO₂CCH(OH)CH(OH)CO₂Na (sodium tartrate); NaOCOCH(OH)CH(OH)COOK (potassium sodium tartrate); CH₂(COONa)₂ (disodium malonate); KCl (potassium chloride);K(OH) (potassium hydroxide); KHCO₃ (potassium bicarbonate); K₂[CO₃](potassium carbonate); KC₆H₇O₆ (potassium ascorbate); K₃C₆H₅O₇ (potassium citrate); KH (potassium hydride); KHSO₄ (potassium hydrogen sulfate); K₂SO₄ (potassium sulfate); K₃PO₃ (potassium phosphate); KHPO₃ (potassium dihydrogen phosphate); K₂PO₃ (potassium hydrogen phosphate); KOCOCH(OH)CH(OH)COOH (monobasic potassium tartrate); KOCOCH(OH)CH(OH)COK (potassium tartrate); KOCOCH(OH)CH(OH)COONa (potassium sodium tartrate); MgCl₂ (magnesium chloride); MgO (magnesium oxide); Mg(OH)₂ (magnesium hydroxide); MgH₂ (magnesium hydride); Mg[HCO₃]₂ (magnesium bicarbonate); MgCO₃ (magnesium carbonate); Mg₃[C₆H₅O₇]₂ (magnesium citrate); MgC₁₀H₁₂N₂O₆ (magnesium pidolate); MgSO₄ (magnesium sulfate); Mg(HSO₄)₂ (magnesium hydrogen sulfate); CaCl₂) (calcium chloride); CaO (calcium oxide); Ca(OH)₂ (calcium hydroxide); CaH₂ (calcium hydride); Ca[HCO₃]₂ (calcium bicarbonate); CaCO₃ (calcium carbonate); Ca₃[C₆H₅O₇]₂ (calcium citrate); CaSO₄ (calcium sulfate); Ca(HSO₄)₂ (calcium hydrogen sulfate); Ca₃(PO₄)₂ (calcium phosphate); CaHPO₄ (calcium hydrogen phosphate); Ca(H₂PO₄)₂ (calcium dihydrogen phosphate); MnCl₂ (manganese chloride); MnO (manganese oxide); Mn(OH)₂ (manganese hydroxide); Mn(SO₄) (manganese sulfate); Mn[HCO₃]₂ (manganese hydrogen carbonate); MnCO₃ (manganese carbonate); Mn₃[C₆H₅O₇]₂ (manganese citrate); Mn₃ (PO₄)₂ (manganese phosphate); and mixtures thereof.

4. The aqueous solution of claim 1, wherein the at least one salt is a monohydrogen carbonate, a bicarbonate or a chloride of an alkaline or alkaline earth metal.

5. The aqueous solution of claim 1, wherein the at least one salt is dihydrogen carbonate or a chloride of an alkaline metal.

6. The aqueous solution of claim 4 wherein the at least one salt is selected from the group consisting of sodium bicarbonate, potassium carbonate, magnesium chloride and a mixture thereof.

7. The aqueous solution of claim 1, wherein the at least one salt is present in the aqueous solution in an amount in the range from 10 mM to 800 mM.

8. The aqueous solution of claim 1, wherein the ascorbic acid or a salt thereof is in an amount in the range from 10 mM to 300 mM.

9. The aqueous solution of claim 1, wherein the ionic strength is in the range from 0.0075 mol/L to 0.5 mol/L.

10. The aqueous solution of claim 1, wherein the aqueous solution comprises also vitamins and/or essential oils.

11. The aqueous solution of claim 1, wherein the aqueous solution has a pH value in the range from 6 to 11.

12. A cosmetic method for ameliorating the activation of cell mechanisms that reduce cellular oxidative stress and cell toxicity comprising the step of administering the aqueous solution of claim 1 by transdermal administration to a subject.

13. The cosmetic method of claim 12, wherein the subject is a subject practicing a sport activity.

14. A method for treating oxidative stress-related diseases by reducing cellular oxidative stress and reducing cell toxicity, comprising the step of administering the aqueous solution of claim 1 by transdermal administration.

15. An apparatus for carrying out the method according to claim 12, comprising
means for generating hydrogen;
one or a plurality of use stations, arranged in proximity and/or remotely with respect to said means for generating hydrogen,
each one including one or a plurality of dipping vessels, suitable for dipping the whole or parts of the body of a human subject, including hands and/or feet, or of an animal subject, suitable for being loaded with water;
means for conveying input load water into said one or each one of said plurality of dipping vessels;
means for conveying output unloading water from said one or each one of said plurality of dipping vessels;
means for transporting output hydrogen from said means for generating hydrogen, arranged for introducing the hydrogen into said water loaded into said one or each one of said plurality of dipping vessels, generating a hydrogen aqueous solution therein;
one or a plurality of means for dispensing at least one salt;
conveyor means arranged for collecting the salts dispensed therefrom and conveying them to said one or each one of said plurality of dipping vessels.

16. The aqueous solution of claim 2, wherein the molecular hydrogen is present in the solution in an amount (concentration) in the range from 0.4 ppm (mg/L) to 0.9 ppm (mg/L).

17. The aqueous solution of claim 7, wherein the at least one salt is present in the aqueous solution in an amount in the range from 50 mM to 400 mM.
